SEQUENCE LISTING (29 sequences)

SEQ ID NO: 1 - protein, 4 aa, Artificial Sequence
  Description of Artificial Sequence Synthetic peptide
LQGV

SEQ ID NO: 2 - protein, 4 aa, Artificial Sequence
  Description of Artificial Sequence Synthetic peptide
AQGV

SEQ ID NO: 3 - protein, 4 aa, Artificial Sequence
  Description of Artificial Sequence Synthetic peptide
LQGA

SEQ ID NO: 4 - protein, 6 aa, Artificial Sequence
  Description of Artificial Sequence Synthetic peptide
VLPALP

SEQ ID NO: 5 - protein, 6 aa, Artificial Sequence
  Description of Artificial Sequence Synthetic peptide
ALPALP

SEQ ID NO: 6 - protein, 6 aa, Artificial Sequence
  Description of Artificial Sequence Synthetic peptide
VAPALP

SEQ ID NO: 7 - protein, 7 aa, Artificial Sequence
  Description of Artificial Sequence Synthetic peptide
ALPALPQ

SEQ ID NO: 8 - protein, 7 aa, Artificial Sequence
  Description of Artificial Sequence Synthetic peptide
VLPAAPQ

SEQ ID NO: 9 - protein, 7 aa, Artificial Sequence
  Description of Artificial Sequence Synthetic peptide
VLPALAQ

SEQ ID NO: 10 - protein, 4 aa, Artificial Sequence
  Description of Artificial Sequence Synthetic peptide
LAGV

SEQ ID NO: 11 - protein, 6 aa, Artificial Sequence
  Description of Artificial Sequence Synthetic peptide
VLAALP

SEQ ID NO: 12 - protein, 6 aa, Artificial Sequence
  Description of Artificial Sequence Synthetic peptide
VLPALA

SEQ ID NO: 13 - protein, 7 aa, Artificial Sequence
  Description of Artificial Sequence Synthetic peptide
VLPALPQ

SEQ ID NO: 14 - protein, 7 aa, Artificial Sequence
  Description of Artificial Sequence Synthetic peptide
VLAALPQ

SEQ ID NO: 15 - protein, 7 aa, Artificial Sequence
  Description of Artificial Sequence Synthetic peptide
VLPALPA

SEQ ID NO: 16 - protein, 7 aa, Artificial Sequence
  Description of Artificial Sequence Synthetic peptide
GVLPALP

SEQ ID NO: 17 - protein, 13 aa, Artificial Sequence
  Description of Artificial Sequence Synthetic peptide
LQGVLPALPQVVC

SEQ ID NO: 18 - protein, 13 aa, Artificial Sequence
  Description of Artificial Sequence Synthetic peptide
LPGCPRGVNPVVS

SEQ ID NO: 19 - protein, 4 aa, Artificial Sequence
  Description of Artificial Sequence Synthetic peptide
LPGC

SEQ ID NO: 20 - protein, 4 aa, Artificial Sequence
  Description of Artificial Sequence Synthetic peptide
MTRV

SEQ ID NO: 21 - protein, 10 aa, Artificial Sequence
  Description of Artificial Sequence Synthetic peptide
VLPALPQVVC

SEQ ID NO: 22 - protein, 10 aa, Artificial Sequence
  Description of Artificial Sequence Synthetic peptide
LQGVLPALPQ

SEQ ID NO: 23 - protein, 8 aa, Artificial Sequence
  Description of Artificial Sequence Synthetic peptide
GVLPALPQ

SEQ ID NO: 24 - protein, 38 aa, Artificial Sequence
  Description of Artificial Sequence Synthetic peptide
VVCNYRDVRFESIRLPGCPRGVNPVVSYAVALSCQCAL

SEQ ID NO: 25 - protein, 35 aa, Artificial Sequence
  Description of Artificial Sequence Synthetic peptide
RPRCRPINATLAVEKEGCPVCITVNTTICAGYCPT

SEQ ID NO: 26 - protein, 18 aa, Artificial Sequence
  Description of Artificial Sequence Synthetic peptide
SKAPPPSLPSPSRLPGPS

SEQ ID NO: 27 - protein, 16 aa, Artificial Sequence
  Description of Artificial Sequence Synthetic peptide
SIRLPGCPRGVNPVVS

SEQ ID NO: 28 - DNA, 25 bp, Artificial Sequence
  Description of Artificial Sequence Synthetic probe
agctcagagggggactttccgagag

SEQ ID NO: 29 - protein, 4 aa, Artificial Sequence
  Description of Artificial Sequence Synthetic peptide
QVVC

(19) United States
(12) Patent Application Publication
(10) Pub. No.: US 2003/0220260 A1
(43) Pub. Date: Nov. 27, 2003

(54) Peptide compositions

(76) Inventors: Nisar Ahmed Khan, Rotterdam (NL); Robbert Benner, Barendrecht (NL)

Correspondence Address: TRASK BRITT, P.O. BOX 2550, SALT LAKE CITY, UT, 84110, US

(21) Appl. No.: 10/409,659

(22) Filed: Apr. 8, 2003

Related U.S. Application Data
(63) Continuation-in-part of application No. 10/028,075, filed on Dec. 21, 2001.

(51) Int. Cl. A61K 38/17; A61K 9/70

(52) U.S. Cl. 514/12; 424/449

(57) ABSTRACT

The invention relates to the treatment of disease wherein a gene-regulatory peptide as provided herein is useful as a modulator of NF-κB. The invention also provides a pharmaceutical composition for topical application comprising a gene-regulatory peptide or functional analogue thereof, wherein the peptide or analogue modulates translocation and/or activity of a gene transcription factor.

### CROSS-REFERENCE TO RELATED APPLICATION

This application is a continuation-in-part of the earlier U.S. patent application Ser. No. 10/028,075, filed Dec. 21, 2001, pending, the content of the entirety of which is incorporated by this reference.

### TECHNICAL FIELD

The current invention relates to the body's innate way of modulation of important physiological processes and builds on insights reported in PCT International Publications WO99/59617 and WO01/72831 and PCT International Patent Application PCT/NL02/00639 (designating the United States of America), the contents of the entirety of all of which are incorporated by this reference.

### BACKGROUND

In the aforementioned patent applications, small gene-regulatory peptides are described that are present naturally in pregnant women and are derived from proteolytic breakdown of placental gonadotropins such as human chorionic gonadotropin (hCG) produced during pregnancy. These peptides (in their active state often only at about 4 to 6 amino acids long) were shown to have unsurpassed immunological activity that they exert by regulating expression of genes encoding immunomodulatory and inflammatory mediators such as cytokines. Surprisingly, it was found that breakdown of hCG provides a cascade of peptides that help maintain a pregnant woman's immunological homeostasis. These peptides are nature's own substances that balance the immune system to assure that the mother stays immunologically sound while her fetus does not get prematurely rejected during pregnancy but instead is safely carried through its time of birth.

Where it was generally thought that the smallest breakdown products of proteins have no specific biological function on their own (except to serve as antigen for the immune system), from the above three patent applications, it now emerges that the body in fact routinely utilizes the normal process of proteolytic breakdown of the proteins it produces to generate important gene-regulatory compounds, short peptides that control the expression of the body's own genes. Apparently the body uses a gene-control system ruled by small broken down products of the exact proteins that are encoded by its own genes.

It has been known that during pregnancy the maternal system introduces a status of temporary immuno-modulation which results in suppression of maternal rejection responses directed against the fetus. Paradoxically, during pregnancy, often the mother's resistance to infection is increased and she is found to be better protected against the clinical symptoms of various auto-immune diseases such as rheumatism and multiple sclerosis. The protection of the fetus can thus not be interpreted only as a result of immune suppression. Each of the above three applications have provided insights by which the immunological balance between protection of the mother and protection of the fetus can be understood.

It was shown that certain short breakdown products of hCG (i.e., short peptides which can easily be synthesized, if needed modified, and used as pharmaceutical composition) exert a major regulatory activity on pro- or anti-inflammatory cytokine cascades that are governed by a family of crucial transcription factors, the NFκB family which stands central in regulating the expression of genes that shape the body's immune response.

Most of the hCG produced during pregnancy is produced by cells of the placenta, the exact organ where cells and tissues of mother and child most intensely meet and where immuno-modulation is most needed to fight off rejection. Being produced locally, the gene-regulatory peptides which are broken down from hCG in the placenta immediately balance the pro- or anti-inflammatory cytokine cascades found in the no-mans land between mother and child. Being produced by the typical placental cell, the trophoblast, the peptides traverse extracellular space; enter cells of the immune system and exert their immuno-modulatory activity by modulating NFκB-mediated expression of cytokine genes, thereby keeping the immunological responses in the placenta at bay.

### BRIEF SUMMARY OF THE INVENTION

It is herein postulated that the beneficial effects seen on the occurrence and severity of auto-immune disease in the pregnant woman result from an overspill of the hCG-derived peptides into the body as a whole; however, these effects must not be overestimated, as it is easily understood that the further away from the placenta, the less immuno-modulatory activity aimed at preventing rejection of the fetus will be seen, if only because of a dilution of the placenta-produced peptides throughout the body as a whole. However, the immuno-modulatory and gene-regulatory activity of the peptides should by no means only be thought to occur during pregnancy and in the placenta; man and women alike produce hCG, for example in their pituitaries, and nature certainly utilizes the gene-regulatory activities of peptides in a larger whole.

Consequently, a novel therapeutic inroad is provided, using the pharmaceutical potential of gene-regulatory peptides and derivatives thereof. Indeed, evidence of specific-up- or down-regulation of NFκB driven pro- or anti-inflammatory cytokine cascades that are each, and in concert, directing the body's immune response was found in silico in gene-arrays by expression profiling studies, in vitro after treatment of immune cells and in vivo in experimental animals treated with gene-regulatory peptides. Also, considering that NF-κB is a primary effector of disease (A. S. Baldwin, J. Clin. Invest., 2001, 107:3-6), using the hCG derived gene-regulatory peptides offer significant potential for the treatment of a variety of human and animal diseases, thereby tapping the pharmaceutical potential of the exact substances that help balance the mother's immune system such that her pregnancy is safely maintained.

### DETAILED DESCRIPTION OF THE INVENTION

PCT International Patent Application PCT/NL02/00639 also provides an insight in the biology and physiology of the nature of regulatory factors in gene regulation in cellular organisms that allows for the identification and development of an artificial or synthetic compound acting as a gene regulator, and its use as new chemical entity for the production of a pharmaceutical composition or its use in the treatment of disease. Many of small peptides are herein identified as short, gene regulatory peptides. These short, gene regulatory peptides are commonly from 2 to 15 amino acids long, but preferably shorter, e.g., from 3 to 12 amino acids, i.e., 4, 5, 6 or 7 amino acids long and are derivable by proteolytic breakdown of endogenous proteins of an organism, or are derivable by proteolytic breakdown of proteins of a pathogen, i.e., during the presence of the pathogen in a host organism, and act as a gene-regulatory peptide to cells of the organism, in that they can exert an often very specific gene regulatory activity on cells of the organism. In a particular embodiment, PCT/NL02/00639 provides specific gene-regulatory peptides and mechanisms allowing for therapeutically controlling NFκB-initiated. gene expression, and thereby modulating pro- and anti-inflammatory cytokine expression under a variety of different conditions and circumstances. Classically, many genes are regulated not by a gene-regulatory peptide that enters the cells but by molecules that bind to specific receptors on the surface of cells. Interaction between cell-surface receptors and their ligands can be followed. by a cascade of intracellular events including variations in the intracellular levels of so-called second messengers (diacylglycerol, Ca²⁺, cyclic nucleotides). The second messengers in turn lead to changes in protein phosphorylation through the action of cyclic AMP, cyclic GMP, calcium-activated protein kinases, or protein kinase C, which is activated by diaglycerol. Many of these classic responses to binding of ligands to cell-surface receptors are cytoplasmic and do not involve immediate gene activation in the nucleus. Some receptor-ligand interactions, however, are known to cause prompt nuclear transcriptional activation of a specific and limited set of genes. However, progress has been slow in determining exactly how such activation is achieved. In a few cases, the transcriptional proteins that respond to cell-surface signals have been characterized.

One of the clearest examples of activation of a pre-existing inactive transcription factor following a cell-surface interaction is the nuclear factor (NF)-κB, which was originally detected because it stimulates the transcription of genes encoding immunoglobulins of the κ class in B-lymphocytes. The binding site for NK-κB in the κ gene is well defined (see for example P. A. Baeuerle and D. Baltimore, 1988, Science 242:540), providing an assay for the presence of the active factor. This factor exists in the cytoplasm of lymphocytes complexed with an inhibitor. Treatment of the isolated complex in vitro with mild denaturing conditions dissociates the complex, thus freeing NF-κB to bind to its DNA site. Release of active NF-κB in cells is now known to occur after a variety of stimuli including treating cells with bacterial lipopolysaccharide (LPS) and extracellular polypeptides as well as chemical molecules (e.g., phobol esters) that stimulate intracellular phosphokinases. Thus a phosphorylation event triggered by many possible stimuli may account for NF-κB conversion to the active state. The active factor is then translocated to the cell nucleus to stimulate transcription only of genes with a binding site for active NF-κB. In PCT/NL02/00639, it was shown that a variety of the short peptides as indicated above exert a modulatory activity on NF-κB activity.

The inflammatory response involves the sequential release of mediators and the recruitment of circulating leukocytes, which become activated at the inflammatory site and release further mediators (Nat. Med. 7:1294; 2001). This response is self-limiting and resolves through the release of endogenous anti-inflammatory mediators and the clearance of inflammatory cells. The persistent accumulation and activation of leukocytes is a hallmark of chronic inflammation. Current approaches to the treatment of inflammation rely on the inhibition of pro-inflammatory mediator production and of mechanisms that initiate the inflammatory response. However, the mechanisms by which the inflammatory response resolves might provide new targets in the treatment of chronic inflammation. Studies in different experimental models of resolving inflammation have identified several putative mechanisms and mediators of inflammatory resolution.

Considering that NF-κB is thought by many to be a primary effector of disease (A. S. Baldwin, J. Clin. Invest., 2001, 107:3-6), numerous efforts are herein provided to develop safe modulators of NF-κB to be used in treatment of both chronic and acute and systemic and local disease situations.

The administration of a gene-regulatory peptide may be done as a single dose, as a discontinuous sequence of various doses, or continuously for a period of time sufficient to permit substantial modulation of gene expression. In the case of a continuous administration, the duration of the administration may vary depending upon a number of factors which would readily be appreciated by those skilled in the art. A gene-regulatory peptide as provided herein is useful as a modulator of NF-κB to be used in treatment of both chronic and acute and systemic and local disease situations.

The administration dose of the gene-regulatory peptide may be varied over a fairly broad range. The concentrations of an active molecule which can be administered would be limited by efficacy at the lower end and the solubility of the compound at the upper end. The optimal dose or doses for a particular patient should and can be determined by taking into consideration relevant factors such as the condition, weight and age of the patient, and other considerations of the physician or medical specialist involved.

The invention is further explained by the use of the following illustrative examples.

### EXAMPLES

One example wherein a gene-regulatory peptide as provided herein is useful as a modulator of NF-κB to be used in treatment of disease is in topical applications. In one embodiment, the invention provides a pharmaceutical composition for topical application comprising a gene-regulatory peptide or functional analogue thereof, and use of a gene-regulatory peptide or functional analogue thereof for the production of a pharmaceutical composition for topical application. Such a composition is most useful to treat a particular surface area, such as a certain area of the skin or mucus membranes and thereby affects essentially only the area to which it is applied and some of the underlying tissue. Where the body logically responds to local insults by eliciting a (local) inflammation, the invention provides use of a gene-regulatory peptide comprising an NF-κB down-regulating peptide or functional analogue thereof for the production of a pharmaceutical composition for the topical treatment of a subject, to actually counter the inflammation and prevent systemic responses and overly active scar tissue formation. The invention also provides a cream or ointment comprising an NF-κB down-regulating peptide or functional analogue thereof, and if so desired a bactericidal or bacteriostatic compound or compound comprising silver. Wound management will vary according to the depth of the insult. The true depth of the affected area will become more obvious with time and therefore the wound must be reassessed to ensure that wound management is appropriate. Systemic, and even topical, antibiotics are not to be used prophylactically, and are in general only appropriate when demonstrated infection is present, however, is in particular useful that translocation and/or activity of the NF-κB/Rel protein is inhibited to counter the local cytokine cascade leading to an inflammation by the inclusion of one or more of the NFκB down-regulating peptides or functional analogues thereof as identified herein, at a concentration of for example 1 to 1000 microg/g, preferably 50-300 microg/g, and at that time it is even more useful that the pharmaceutical composition for topical use is also provided with antibacterial compounds, such as compounds that comprise silver, such as a antibacterial cream or ointment comprising micronized silver sulfadiazine and an NFκB down-regulating peptide. The invention thus provides a method to treat an injury of a subject wherein the subject is provided with a topical agent directed against a bacterial infection such as a bacteriostatic or bactericidal compound such as tetracycline or a sulfa compound wherein the topical agent also comprises an NFκB down-regulating peptide at a concentration of for example 1 to 1000 microg/g, preferably 50-300 microg/g. Typical other substances found in such a cream or ointment are 10 mg/gram of micronized silver sulfadiazine and a lege artis cream. vehicle composed of white petrolatum, stearyl alcohol, isopropyl myristate, sorbitan monooleate, polyoxyl 40 stearate, propylene glycol, and water. Another anti-inflammatory and anti-infective; cream for topical administration as herein provided comprises one or more of NFκB down-regulating peptides VLPALPQVVC, LQGVLPALPQ, LQG, LQGV, GVLPALPQ, VLPALP, VVC, MTR at a concentration of for example 50-300 microgram/gram and contains per gram mafenide acetate equivalent to 85 mg of the base. The cream vehicle for example consists of cetyl alcohol, stearyl alcohol, cetyl esters wax, polyoxyl 40 stearate, polyoxyl 8 stearate, glycerin, and water, with methylparaben, propylparaben, sodium metabisulfite, and edetate disodium as preservatives may be added.

Other useful pharmaceutical compositions for topical use are an oil-in-water emulsion base composed of glycerin, cetyl alcohol, stearic acid, glyceryl monostearate, mineral oil, polyoxyl 40 stearate, menthol, benzyl alcohol,- and purified water, comprising a gene-regulatory peptide, preferably an NF-κB down-regulating peptide at a concentration of for example 50-300 microgram/gram, a hydrophilic, emollient cream base of propylene glycol, white petrolatum, cetearyl alcohol, glyceryl stearate, PEG 100 stearate, monobasic sodium phosphate, chlorocresol, phosphoric acid, and purified water with gene-regulatory peptides at the above preferably concentrations, an ointment base of hexylene glycol, white wax, propylene glycol stearate, and white petrolatum with the peptides; an emollient cream base of purified water, chlorocresol; propylene glycol, white petrolatum, white wax, cyclomethicone; sorbitol solution, glyceryl oleate/propylene glycol, with gene-regulatory peptide; a lotion base of purified water, isopropyl alcohol, hydroxypropyl cellulose, propylene glycol, sodium phosphate monobasic monohydrate, phosphoric acid, preferably used to adjust the pH to 4.5, and gene regulatory peptide; polyethylene glycol ointment, and gene-regulatory peptide; a topical gel, containing the active ingredient gene-regulatory peptide at 25-250 mg/g and the following inactive ingredients: sodium chloride, sodium acetate trihydrate, glacial acetic acid, water for injection.

In another example wherein a gene-regulatory peptide as provided herein is useful as a modulator of NF-κB to be used in treatment of disease the invention provides a pharmaceutical composition for topical application to the eye or ear comprising a gene-regulatory peptide or functional analogue thereof, and use of a gene-regulatory peptide or functional analogue thereof for the production of a pharmaceutical composition for application to the eye or ear. Such a composition is most useful to treat a surface area, such as the conjunctivae of the eyes, or the lining of the tympanic cavity. In a further embodiment, the invention provides a pharmaceutical composition comprising a gene regulatory peptide or functional equivalent thereof for the treatment of a conjunctivitis, notably for the treatment of a so called dry eye. Dry eye is the result of any number of unrelated causes or conditions. Some of the most common factors that contribute to dry eye include: Dry or dusty environment, aging, hormonal changes, autoimmune diseases such as seen with Sjogren's disease, certain types of medications and contact lens wear. Regardless of the cause, all dry eye patients have in common abnormal or insufficient tears. This leads to reduced tear clearance, increased osmolarity, ocular surface irritation, and the infiltration and production of pro-inflammatory cytokines. The end result is inflammation. Once inflammation starts, damage can occur to ocular structures that perpetuate and intensify a cycle of signs, symptoms and further inflammation. To counter this inflammation, the invention provides a pharmaceutical composition comprising a gene-regulatory peptide for the treatment of dry eye or other conjunctivitis. Such an ophthalmic preparation for example comprises a sterile, isotonic, and buffered to pH 7.4, aqueous solution with gene-regulatory peptide, preferably an NFκB-down-regulating peptide as indicated herein at 10-100 microgram/milliliter. Inactive ingredients are for example monobasic and dibasic sodium phosphate, sodium hydroxide to adjust pH, and water for injection.

In another example wherein a gene-regulatory peptide as provided herein is useful as a modulator of NF-κB to be used in treatment of disease the invention provides a pharmaceutical composition for systemic application, in particular selected from the group pharmaceutical compositions for intravenous, intraperitoneal, intrathoracal, or intramuscular administration, comprising a gene-regulatory peptide or functional analogue thereof, and use of a gene-regulatory peptide or functional analogue thereof for the production of a pharmaceutical composition for systemic application. Such a composition is most useful to treat the body as a whole and in a preferred embodiment thereby affects essentially not or only little the area to which it is applied.

In another example, wherein a gene-regulatory peptide as provided herein is. useful as a modulator of NF-κB to be used in treatment of disease, the invention is providing a method and means to treat the systemic immunosuppressive reaction to trauma or surgery by providing a subject believed to be in need thereof with a pharmaceutical composition for systemic application comprising an NF-κB down-regulating peptide or functional analogue thereof and an agent directed against disseminated intravascular coagulation. Such an agent may be, for example, a composition comprising heparin, however, in a preferred embodiment, the invention provides treatment with a hypotonic pharmaceutical composition for systemic application comprising an NF-κB up-regulating peptide or functional analogue thereof. Such treatment may for example comprise infusions with Ringer's lactate for the first 24 hours, the Ringer's lactate provided with, preferably, 1-1000 mg/l NFκB regulating peptide such as VLPALPQ, GVLPALP or MTRV, or mixtures of two or three of these peptides. During resuscitation, it is often important to keep the volume up, and, if needed, provide the peptide or functional analogue thereof in even further hypotonic solutions, such as in 0.3 to 0.6% saline. NFκB regulating peptide can be given in the same infusion, the peptide (or analogue) concentration preferably being from about 1 to about 1000 mg/l, but the peptide can also been given in a bolus injection. Doses of 1 to 5 mg/kg bodyweight, for example every eight hours in a bolus injection or per infusionem until the patient stabilizes, are recommended. It is preferred to monitor cytokine profiles, such as TNF-α or IL-10 levels, in the plasma of the treated patient, and to stop treatment when these levels are considered within normal boundaries.

In another example wherein a gene-regulatory peptide as provided herein is useful as a modulator of NF-κB to be used in treatment of disease, it is herein provided to modulate immunosuppression in a traumatized subject comprising providing the subject with a gene-regulatory peptide comprising a gene-regulatory peptide or functional analogue thereof wherein the subject is also provided with an agent directed against disseminated intravascular coagulation, in particular wherein the agent comprises Activated Protein C activity. Such an agent to modulate disseminated intravascular coagulation (DIC) comprises preferably (recombinant) human Activated Protein C. It is preferably given to the patient per infusionem, whereby NFκB regulating peptide can be given in the same infusion, the peptide (or analogue) concentration preferably being from about 1 to about 1000 mg/l, but the peptide can also been given in a bolus injection. Doses of 1 to 5 mg/kg bodyweight, for example every eight hours in a bolus injection or per infusionem until the patient stabilizes, are recommended.

In another example wherein a gene-regulatory peptide as provided herein is useful as a modulator of NF-κB to be used in treatment of disease, the invention also provides a hypertonic pharmaceutical composition, such as a resuscitation fluid comprising an NF-κB down-regulating peptide or functional analogue thereof. For example, when the subject is in immediate need of a peptide solution to fence of ischemia-reperfusion damage, but time is too short or valuable to give this solution in saline, considering the volume required, it is herein provided to use a hypertonic resuscitation fluid, such as a hypertonic salt solution provided with one or more of the herein mentioned NFκB regulatory peptides at a concentration of 1 to 1000 mg/l. Administration of hypertonic saline (HS) with gene-regulatory peptide to a for example a victim of a traffic accident intravenously causes an initial rapid fluid influx into the vasculature. This is due to the sudden hypertonic state of plasma caused by the infusion of HS (for example 7.5%, 1283 mmol/l NaCl) in a relatively short time. Other useful sodium concentrations range from 1.2% to 10%. Water is shifted from the intracellular spaces, first the erythrocytes and endothelial cells and then from the tissue cells, into the extracellular compartment. Shrinkage of the endothelium has also beneficial microcirculatory effects due to the reduced resistance of the capillaries. Interstitial water- also moves into the intravascular compartment by the osmotic gradient. Hypertonic saline expands intravascular volume by mobilizing fluid that is already present in the body; intracellular and interstitial fluid is shifted into the intravascular space. Plasma volume expansion is therefore with less free water administration than with isotonic plasma expanders. The effect of the hypertonic peptide solution on plasma volume is transient since the fluid will shift from the intravascular space back to the extravascular space, allowing a rapid and thoroughly systemic distribution of a gene-regulatory, peptide in the body. Other circumstances wherein hypertonic solutions as provided herein are useful are for giving immediate, —out-of-hospital—care, such as in an ambulance or at the battlefield, after cardiac attacks and before the patient is transferred to an intensive care unit, and other emergencies where immediate care is needed. Other useful hypertonic solutions may be prepared as well: such as hypertonic NaCl (2400 mosM), hypertonic glucose (2400 mosM), hypertonic sorbitol (2400 mosM), hypertonic glucose (1200 mosM)/glycine (1200 mosM), hypertonic glucose. (600 mosM)/ mannitol (600 mosM)/glycine (1200 mosM), and hypertonic sorbitol (1200 mosM)/glycine (1200 mosM), each provided with gene-regulatory peptide at 1 to 1000 mg/l. In particular, it is herein provided to use these hypertonic solutions with gene-regulatory peptide initially as a small volume (4-5 ml/kg) infusion fluid. A 80-kg man should receive for example 320-400 ml hypertonic solution with 1-5 mg peptide or functional equivalent/kg body weight. Taking into account an average blood volume of 6 L (6000 ml) and a hematocrit of 45%, the small volume of HS will be distributed into approximately 3300 ml cellular free blood volume. This corresponds to an increase of the cellular free volume of approximately 3620-3700 ml. This also corresponds to approximately 9-11% plasma volume replacement. Initially, for every ml of hypertonic solution with gene regulatory peptide infused, about 7 ml of free water is drawn into the blood stream. Then, once the peptide is distributed and equilibrium is reached, an additional 2240-2800 ml free water will be available in the vascular system. This will cause an expansion of the cellular free blood volume, which will reach about 5860-6500 ml. Thus, under equilibrium conditions, approximately 5-6% of the plasma volume will be replaced, by hypertonic solution with gene-regulatory peptide. Then, reperfusion of the subject can be continued with normal (i.e. isotonic) reperfusion fluid, such as Ringer's lactate, or even hypotonic solutions of for example hypotonic saline. This results in an expansion of intracellular volume, further facilitating entry of gene-regulatory peptide.

In another example wherein a gene-regulatory peptide as provided herein is useful as a modulator of NF-κB to be used in treatment of disease the invention provides combination therapy that include the concomitant treatment of the patient with a (monoclonal) antibody directed against a cytokine, such as TNF-α, IL-6 or IL-12, However, although few would disagree that using these cytokine-blocking agents such as anti-TNF-α therapy may be an important therapeutic addition in the treatment of patients with inflammations such as seen with IBDS, in particular with Crohn's disease, adverse effects related to single cytokine neutralizing therapies have emerged. Also, for unknown reasons, single cytokine blocking proteins may cause the formation of anti-dsDNA antibodies, and after repeated treatment the cumulative ANA incidence can be as high as 50%. Nonetheless, anti-TNF-α antibody therapy is associated with lupus-like symptoms. Also, demyclinizing disease and aplastic anemia have been reported in a small number of thus treated patients. A major problem of repeated administration of chimeric therapeutic antibodies is immunogenicity, and up to 60% of antibody treated patients develop human antichimeric antibodies (HACAs) which are related to infusion reactions and reduce therapeutic efficacy. In comparison with single cytokine therapy, such as the use of anti-TNF-α, anti IL-5, anti-IL-6, anti-IL-12, anti-IL-23, anti-IL-12p40, anti-IL23p40 or anti-IL-1β antibodies, using an NFκB down-regulating peptide or functional analogue thereof according to the invention has the major advantage that a major network of pro-inflammatory cytokines is down-regulated.

In another embodiment, it is herein provided to modulate immunosuppression in. a traumatized subject comprising providing the subject with a gene-regulatory peptide comprising a gene-regulatory peptide or functional analogue thereof wherein the subject is also provided with an agent directed against disseminated intravascular coagulation, in particular wherein the agent comprises Activated Protein C activity. Such an agent to modulate disseminated intravascular coagulation (DIC) comprises preferably (recombinant) human Activated Protein C. It is preferably given to the patient per infusionem, whereby NFκB regulating peptide can be given in the same infusion, the peptide (or analogue) concentration preferably being from about 1 to about 1000 mg/l, but the peptide can also been given in a bolus injection. Doses of 1 to 5 mg/kg bodyweight, for example every eight hours in a bolus injection or per infusionem until the patient stabilizes, are recommended.

In another example wherein a gene-regulatory peptide as provided herein is useful as a modulator of NF-κB to be used in treatment of disease, the invention provides use of an NFκB regulating peptide or derivative thereof for the production of a pharmaceutical composition for the treatment of inflammatory disease in a primate, and provides a method of treatment of inflammatory disease in a primate. It is preferred that the, treatment comprises administering to the subject a pharmaceutical composition comprising an NFκB down-regulating peptide or functional analogue thereof. Examples of useful NFκB down-regulating peptides are VLPALPQVVC, LQGVLPALPQ, LQG, LQGV, GVLPALPQ, VLPALP, VVC, MTR and circular LQGVLPALPQVVC. More down-regulating peptides and functional analogues can be found using the methods as provided herein. Most prominent among NFκB down-regulating peptides are VLPALPQVVC, LQGVLPALPQ, LQG, LQGV, and VLPALP. These are also capable of reducing production of NO by a cell. In one embodiment, the invention provides a method of treating a subject suffering from an inflammatory disease with a method and gene-regulatory peptide according to the invention concomitantly, or at least timely, with a treatment with a single cytokine blocking protein, such as an anti-TNF-α, anti IL-5, anti-IL-6, anti-IL-12, anti-IL-23, anti-IL-12p40, anti-IL23p40 or anti-IL-1βantibody or functional analogue thereof It is herein also provided to use a gene-regulatory peptide according to the invention for the production of a pharmaceutical composition for the treatment of a subject believed to be suffering of an inflammation and receiving treatment with an anti-cytokine antibody such as an anti-TNF-α, anti IL-5, anti-IL-6, anti-IL-12, anti-IL-23, anti-IL-12p40, anti-IL23p40 or anti-IL-1βantibody. It is herein also provided to use a composition that comprises at least two oligopeptides or functional analogues thereof, each capable of down-regulation NFκB, and thereby reducing production of NO and/or TNF-α by a cell, in particular wherein at least two oligopeptides are selected from the group LQGV, AQGV and VLPALP, for the treatment of inflammatory diseased patients that are also treated with an anti-cytokine antibody. In a particular example wherein, a gene-regulatory peptide as provided herein is useful as a modulator of NF-κB to be used in treatment of disease, such treatment as provided herein is applicable to patients suffering from inflammatory bowels disease (IBD). Inflammatory bowel disease (IBD) is a chronic relapsing and remitting inflammatory condition of the gastrointestinal tract that is manifest as 1 of 2 usually distinct but sometimes overlapping clinical entities, ulcerative colitis (UC) and Crohn disease (CD). Ulcerative colins affects the colon and is a superficial ulcerative disease, whereas CD is a transmural granulomatous disorder that affects any part of the gastrointestinal tract and has a predilection for the terminal ileum and colon. Both forms of IBD are associated with prominent extra-intestinal manifestations and an increased incidence of gastrointestinal cancer, in addition, both begin relatively early in life and persist for long periods, leading to decreased quality of life indices and a greater than 2-fold increase in mortality rate.

By and large, IBD is a disease of “urbanized” areas such as the United States and Europe, where it occurs at an incidence of 6 to 12 and 5 to 7 per 100,000 population for UC and CD, respectively. This translates to 45.000 new cases per year and 1 million affected individuals in the United States alone and costs the US health care system approximately $ 1.8 billion per year (1990 estimate).

The concept that the proximal cause of IBD is immunologic in nature arose from the observation that IBD is characterized by massive cellular infiltrates and is associated with abnormalities of the immune system that include NFκB induced inappropriate production of antibodies and T-cell dysfunctions. This concept has been clarified by studies of patient lamina propria (LP) cells that show that in Crohn's disease (CD), NFκB induces immune cells to overproduce cytokines indicative of a typical helper T-cell 1 (T_{H}1) response, namely increased production of interleukin (IL) 12 by LP macrophages and increase production of interferon (IFN) γ by LP T cells. In addition, LP T cells from patients with ulcerative colitis (UC) manifest a cytokine profile compatible with a T_{H}2 response; thus, while the cells do not overproduce the major T_{H}2 cytokine IL-4, they do produce increased amounts of another T_{H}2 cytokine, IL-5. This cytokine production pattern accords with an association of UC (but not CD) with auto-antibodies that in general require T_{H}2 responses.

These data provide evidence that the 2 major forms of IBD are due to misregulated or excessive T_{H}1 (CD) or T_{H}2 (UC) responses. As to what factors induce these abnormal responses, there is considerable evidence that IBD patients have inappropriate T-cell responses to antigenic components of their own intestinal microflora, that benefit from treatment with a gene-regulatory peptide as provided herein to serve as a modulator of NF-κB to be used in treatment of IBD both because of the beneficial effects on the earlier dysfunction in the primary or secondary mechanisms that normally drive and regulate such response and because of down-regulation of the inflammatory cascade in the intestinal epithelial cell barrier that otherwise leads to inappropriate penetration of microbial antigens.

In another example wherein a gene-regulatory peptide as provided herein is useful as a modulator of NF-κB to be used in treatment of disease, the invention provides treatment with a gene-regulatory peptide concomitantly with selective decontamination of the gut, as practiced in some patients, for example in preparation of a patient for a bone marrow transplantation, induces additional pro-inflammatory cytokine release, which can add to the pro-inflammatory burst in case of a complication such as hemorrhagic shock. Selective decontamination of the gut, as practiced in some patients, for example in preparation of a bone marrow transplant, induces additional proinflammatory cytokine release, which can add to the proinflammatory burst in case of a complication such as hemorrhagic shock. Also, major surgery, such as cardiopulmonary bypass predisposes the splanchnic region to inadequate perfusion and increases in gut permeability. Related to these changes, circulating endotoxin has been shown to rise during surgery, and contributes to cytokine activation, high oxygen consumption, and fever (“post-perfusion syndrome”). To a large extent, free endotoxin in the gut is a product of the proliferation of aerobic gram-negative bacteria and may be reduced by nonabsorbable antibiotics, however, selective decontamination of the gut does not affect the occurrence of perioperative endotoxemia, nor does it reduce the tumor necrosis factor-α or interleukin-6 concentrations as determined before surgery, upon aorta declamping, 30 minutes into reperfusion, or 2 hours after surgery. Also, selective decontamination of the gut does not alter the incidence of postoperative fever or clinical outcome measures such as duration of artificial ventilation and intensive care unit and hospital stay. In conclusion, treatment with a gene-regulatory peptide as provided herein is useful as a modulator of NF-κB to affect the incidence of perioperative endotoxemia, pro-inflammatory cytokine activation and the occurrence of a post-perfusion syndrome during or after surgery.

In another example wherein a gene-regulatory peptide as provided herein is useful as a modulator of NF-κB to be used in treatment of disease, the invention provides a pharmaceutical composition for the treatment of an overly strong immune response occurring in a primate, and a method for the treatment of an overly strong immune response resulting in additional pro-inflammatory cytokine release in a primate comprising subjecting the subject to a gene-regulatory peptide according to the invention, preferably to a mixture of such gene-regulatory peptides. Administration of such a gene-regulatory peptide or mixture preferably occurs systemically, for example, by intravenous or intraperitoneal administration and leads to a dampening of the effect of the additionally released pro-inflammatory cytokines. In a further embodiment, such treatment also comprises the use of for example an antimicrobial agent, however, especially when such use is otherwise contraindicated or at least considered at risk because of the chance of generating toxin loads that lead to an additional pro-inflammatory cytokine response because of lysis of the microbe subject to the action of those antibiotics in an individual thus treated.

In another example wherein a gene-regulatory peptide as provided herein is useful as a modulator of NF-κB to be used in treatment of disease, the invention provides a mode of treatment of the additional pro-inflammatory cytokine response seen after surgical interventions.

The invention also provides a method for treating a subject that is or has already been treated with another pharmaceutical composition and believed to be suffering or at risk from the side-effects of such a composition or at least believed to profit from the here provided concomitant therapy comprising providing the subject with a gene-regulatory peptide comprising a short, gene regulatory peptide or functional analogue thereof, wherein the gene-regulatory peptide is administered in an amount sufficient to modulate possible side-effects, for example wherein the another pharmaceutical composition is selected from the group of antigens, vaccines, antibodies, anticoagulants, antibiotics, in particular β-lactam antibiotics, antitoxins, antibacterial agents, antiparasitic agents, antiprotozootic agents, antifungal agents, antiviral agents, cytolytic agents, cytostatic agents, thrombolytic agents, and others. In a preferred embodiment, the invention provides a method of treating a subject with a method and gene-regulatory peptide according to the invention concomitantly, or at least timely, with a thrombolytic agent, such as (recombinant) tissue plasminogen activator, or truncated forms thereof having tissue plasminogen activity, or streptokinase, or urokinase. The invention also provides a method for modulating an iatrogenic event in a subject that is concomitantly or already treated with a pharmaceutical composition and believed to be suffering or at risk from the side-effects of such a composition comprising providing the subject with a gene-regulatory peptide comprising a short, gene regulatory peptide or functional analogue thereof, wherein the gene-regulatory peptide is administered in an amount sufficient to modulate the resulting inflammatory cascade, for example wherein the pharmaceutical composition is selected from the group of antigens, vaccines, antibodies, anticoagulants, antibiotics, in particular β-lactam antibiotics, antitoxins, antibacterial agents, antiparasitic agents, antiprotozootic agents, antifungal agents, antiviral agents, cytolytic agents, cytostatic agents, thrombolytic agents. The invention for example provides a method to control the toxic effects of the lysis or damage to bacterial pathogens which release endotoxin and a host of other enterotoxin and exotoxins, resulting in an at times undesirable pro-inflammatory cytokine cascade. Furthermore, the invention provides a method to treat a viral infection. The invention also provides a method wherein the iatrogenic event includes the treatment of a subject with a virus, especially wherein lysis is due to treatment of the subject with the virus. A clear example of the beneficial use of a peptide or functional analogue thereof according to the invention to control a therapy-impeding inflammatory reaction relates to the example of an inflammatory response to (for example adenoviral or retroviral) gene vectors, e.g., in gene therapy such as in treatment of cystic fibrosis. The peptides can be administered systemically as indicated above in the case of cystic fibrosis gene therapy. In another example, the virus comprises a lytic phage used in antibacterial therapy as discussed above.

In another example wherein a gene-regulatory peptide as provided herein is useful as a modulator of NF-κB to be used in treatment of disease, a peptide according to the invention, or a functional derivative or analogue thereof is used for the production of a pharmaceutical composition, for the treatment or mitigation of a pro-inflammatory cytokine response seen after a virus infection. Examples of useful NFκB down-regulating peptides to be included in such a pharmaceutical composition are VLPALPQVVC, LQGVLPALPQ, LQG, LQGV, GVLPALPQ, VLPALP, VVC, MTR and circular LQGVLPALPQVVC, which are for example useful in the treatment of an overly strong IL-6 response seen after severe acute respiratory syndrome (SARS). It is preferred to include concomitant treatment with an antiviral agent such as ribavirine and a gene-regulatory peptide to reduce for example NFκB modulated IL-6 activity as provided herein. More gene-regulating peptides and functional analogues can be found in a (bio)assay, such as an NFκB translocation assay as provided herein. Most prominent among NFκB down-regulating peptides are VLPALPQVVC, LQGVLPALPQ, LQG, LQGV, and VLPALP. These are also capable of reducing production of NO by a cell. It is herein also provided to use a composition that comprises at least two oligopeptides or functional analogues thereof, each capable of down-regulation NFκB, and thereby reducing production of NO and/or TNF-α by a cell, in particular wherein the at least two oligopeptides are selected from the group LQGV, AQGV and VLPALP. Useful NFκB up-regulating peptides are VLPALPQ, GVLPALP and MTRV. As indicated, more gene-regulatory peptides may be founds with an appropriate (bio)assay. A gene-regulatory peptide as used herein is preferably short. Preferably, such a peptide is 3 to 15 amino acids long, and capable of modulating the expression of a gene, such as a cytokine, in a cell. In a preferred embodiment, a peptide is a gene-regulatory peptide that is capable of traversing the plasma membrane of a cell or, in other words, a peptide that is membrane-permeable, more preferably, wherein the lead peptide is 3 to 9 amino acids long, most preferred wherein the gene-regulatory peptide is 4 to 6 amino acids long.

In one embodiment of the present invention, a gene-regulatory peptide is administered in an effective concentration to an animal or human systemically, e.g., by. intravenous, intra-muscular or intraperitoneal administration. Another way of administration comprises perfusion of organs or tissue, be it in vivo or ex vivo, with a perfusion fluid comprising a gene-regulatory peptide according to the invention.

In patients with contusio cerebri and intracranial pressure treatment it is advantageous to combine treatment with the peptides or functional analogues thereof with osmotic agents like mannitol to reduce intracranial pressure and stimulate cerebral perfusion, i.e., by administering intravenous infusions of mannitol 20% in 0.9% saline solutions. of 200 ml, or another hypertonic solution, 1 to 6 times a day. NFκB regulating peptide can be given in the same infusion, the peptide (or analogue) concentration preferably being from about 1 to about 1000 mg/l, but the peptide can also been given in a bolus injection. Doses of 1 to 5 mg/kg bodyweight, for example every eight hours in a bolus injection or per infusionem until the patient stabilizes, are recommended.

In the case of a cardiovascular or cerebrovascular incident, such treatment can for example take the form of intravenous infusions of recombinant tissue plasminogen activator (rt-PA) at a dose of 0.9 mg/kg (maximum of 90 mg) in 0.9% saline solutions, whereby it is preferred that 10% of the rt-PA dose is given within 1 to 2 minutes and the remaining dose of rt-PA in 60 minutes. In the case of an acute myocardial infarction, such treatment can for example take the form of intravenous infusions of rt-PA at a dose of 15 mg as intravenous bolus, followed by 50 mg in the next 30 minutes followed by 35 mg in the next 60 minutes. For the sake of treating the resulting perfuision injury that occurs due to the lysis of the thrombus and the subsequent perfusion of the ischemic area, it is herein provided to also provide the patient with a bolus injection of NF-κB down-regulating peptide such as AQGV, LQGV or VLPALP at 2 mg/kg and continue the infusion with an NF-κB down regulating peptide such as AQGV, LQGV or VLPALP or a functional analogue thereof at a dose of 1 mg/kg bodyweight for every eight hours.

Also of clinical and medical interest and value, the present invention provides the opportunity to selectively control NFκB-dependent gene expression in tissues and organs in a living subject, preferably in a primate, allowing upregulating essentially anti-inflammatory responses such as IL-10, and downregulating essentially pro-inflammatory responses such as mediated by TNF-α, nitric oxide (NO), IL-5, IL-6, IL-12 and IL-1β. For example in comparison with single cytokine therapy, such as the use of anti-TNF-α, anti IL-5, anti-IL-6,-,anti-IL-12 or anti-IL-1β antibodies, using an NFκB down-regulating peptide or functional analogue thereof according to the invention has the distinct advantage that a major network of pro-inflammatory cytokines is down-regulated. It is preferred when the treatment comprises administering to the subject a pharmaceutical composition comprising an NFκB down-regulating peptide or functional analogue thereof. Examples of useful NFκB down-regulating peptides are VLPALPQVVC, LQGVLPALPQ, LQG, LQGV, GVLPALPQ, VLPALP, VVC, MTR and circular LQGVLPALPQWVC. More down-regulating peptides and functional analogues can be found using the methods as provided herein. Most prominent among NFκB down-regulating peptides are VLPALPQVVC, LQGVLPALPQ, LQG, LQGV, and VLPALP. These are also capable of reducing production of NO by a cell. In one embodiment, the invention provides a method of treating a subject suffering from a relapsing/remitting disease seen with MS with a method and gene-regulatory peptide according to the invention concomitantly, or at least timely, with a treatment with a single cytokine blocking protein, such as an anti-TNF-α, anti IL-5,: anti-IL-6, anti-IL-12 or anti-IL-1β antibody or functional analogue thereof. It is herein also provided to use a gene-regulatory peptide according to thee invention for the production of a pharmaceutical composition for the treatment of a subject believed to be suffering of an inflammation, such as Crohn's disease or multiple sclerosis and receiving treatment with an anti-TNF-α, anti IL-5, anti-IL-6, anti-IL-12 or anti-IL-1β antibody. It is herein also provided to use a composition that comprises at least two oligopeptides or functional analogues thereof, each capable of down-regulation NFκB, and thereby reducing production of NO and/or TNF-α by a cell, in particular wherein the at least two oligopeptides are selected from the group LQGV, AQGV and VLPALP, for the treatment of for the treatment of a subject believed to be suffering of an inflammation, such as Crohn's disease or multiple sclerosis and receiving treatment with an anti-TNF-α, anti IL-5, anti-IL-6, anti-IL-12 or anti-IL-1β antibody.

The invention provides a method for modulating transplant survival in a recipient of the transplant comprising providing the transplant with a gene-regulatory peptide comprising a peptide or functional analogue thereof. It is preferred that the peptide is 3 to 15 amino acids long, more preferably, that the peptide is 3 to 9 amino acids long, it most preferred that the peptide is 4 to 6 amino acids long. It is in particular preferred that the gene-regulatory peptide is capable of inhibiting NF-κB/Rel protein activity. Functional analogue herein relates to the signaling molecular effect or activity as for example can be measured by measuring nuclear translocation of a relevant transcription factor, such as. NF-κB in an NF-κB assay, or AP-1 in an AP-1 assay, or by another method as provided herein. Fragments can be somewhat (i.e. 1 or 2 amino acids) smaller or larger on one or both sides, while still providing functional activity. In one embodiment of the invention, the peptide used as a gene-regulatory peptide a chemically modified peptide. A peptide modification includes phosphorylation (e.g., on a Tyr, Ser or Thr residue), N-terminal acetylation, C-terminal amidation, C-terminal hydrazide, C-terminal methyl ester, fatty acid attachment, sulfonation (tyrosine), N-terminal dansylation, N-terminal succinylation, tripalmitoyl-S-Glyceryl Cysteine (PAM3 Cys-OH) as well as farnesylation of a Cys residue. Systematic chemical modification of a peptide can for example be performed in the process of peptide optimization.

Synthetic peptides can be obtained using various procedures known in the art. These include solid phase peptide synthesis (SPPS) and solution phase organic synthesis (SPOS) technologies. SPPS is a quick and easy approach to synthesize peptides and small proteins. The C-terminal amino acid is typically attached to a cross-linked polystyrene resin via an acid labile bond with a linker molecule. This resin is insoluble in the solvents used for synthesis, making it relatively simple and fast to wash away excess reagents and by-products. The peptide, or its functional analogue, modification or derivative, can be administered as the entity as such or as a pharmaceutically acceptable acid- or base-addition salt, formed by reaction with an inorganic acid (such as hydrochloric acid, hydrobromic acid, perchloric acid, nitric acid, thiocyanic acid, sulfuric acid, and phosphoric acid); or with, an organic acid (such as formic acid, acetic acid, propionic acid, glycolic acid, lactic acid, pyruvic acid, oxalic acid, malonic acid, succinic acid, maleic acid, and fumaric acid); or by reaction with an inorganic base (such as sodium hydroxide, ammonium hydroxide, potassium hydroxide); or with an organic base (such as mono-, di-, trialkyl and aryl amines and substituted ethanolamines). A selected peptide and any of the derived entities may also be conjugated to sugars, lipids, other polypeptides, nucleic acids and PNA; and function in-situ as a conjugate or be released locally after reaching a targeted tissue or organ.

In response to a variety of pathophysiological and developmental signals, the NFκB/Rel family of transcription factors is activated and form different types of hetero- and homodimers among themselves to regulate the expression of target genes containing κB-specific binding sites. NF-κB transcription factors are hetero- or homodimers of a family of related proteins characterized by the Rel homology domain. They form two subfamilies, those containing activation domains (p65-RELA, RELB, and c-REL) and those lacking activation domains (p50, p52). The prototypical NFκB is a heterodimer of p65 (RELA) and p50 (NF-κB1). Among the activated NFκB dimers, p50-p65 heterodimers are known to be involved in enhancing the transcription of target genes and p50-p50 homodimers in transcriptional repression. However, p65-p65 homodimers are known for both transcriptional activation and repressive activity against target genes. κB DNA binding sites with varied affinities to different NFB dimers have been discovered in the promoters of several eukaryotic genes and the balance between activated NFκB homo- and heterodimers ultimately determines the nature and level of gene expression within the cell. The term “NFκB-regulating peptide” as used herein refers to a peptide or functional analogue or a modification or derivative thereof capable of modulating the activation of members of the NFκB/Rel family of transcription factors. Activation of NFκB can lead to enhanced transcription of target genes. Also, it can lead to transcriptional repression of target genes. NFκB activation can be regulated at multiple levels. For example, the dynamic shuttling of the inactive NFκB dimers between the cytoplasm and nucleus by IκB proteins and its termination by phosphorylation and proteasomal degradation, direct phosphorylation, acetylation of NFκB factors, and dynamic reorganization of NFκB subunits among the activated NFκB dimers have all been identified as key regulatory steps in NFκB activation and, consequently, in NFκB-mediated transcription processes. Thus, an NFκB-regulating peptide is capable of modulating the transcription of genes that are under the control of NF-κB/Rel family of transcription factors. Modulating comprises the upregulation or the downregulation of transcription.

The term “pharmaceutical composition” as used herein is intended to cover both the active gene-regulatory peptide alone or a composition containing the gene-regulatory peptide together with a pharmaceutically acceptable carrier, diluent or excipient. Acceptable diluents of an oligopeptide as described herein in the detailed description are for example physiological salt solutions or phosphate buffered salt solutions.

In response to a variety of pathophysiological and developmental signals, the NFκB/Rel family of transcription factors is activated and form different types of hetero- and homodimers among themselves to regulate the expression of target genes containing κB-specific binding sites. NF-κB transcription factors are hetero- or homodimers of a family of related proteins characterized by the Rel homology domain. They form two subfamilies, those containing activation domains (p65-RELA, RELB, and c-REL) and those lacking activation domains (p50, p52). The prototypical NFκB is a heterodimer of p65 (RELA) and p50(NF-κB1). Among the activated NFκB dimers, p50-p65 heterodimers are known to be involved in enhancing the transcription of target genes and p50-p50 homodimers in transcriptional repression. However, p65-p65 homodimers are known for both transcriptional activation and repressive activity against target genes. κB DNA binding sites with varied affinities to different NFκB dimers have been discovered in the promoters of several eukaryotic genes and the balance between activated NFκB homo- and heterodimers ultimately determines the nature and level of gene expression within the cell. The term “NFκB-regulating peptide” as used herein refers to a peptide or a modification or derivative thereof capable of modulating the activation of members of the NFκB/Rel family of transcription factors. Activation of NFκB can lead to enhanced transcription of target genes. Also, it can lead to transcriptional repression. of target genes. NFκB activation can be regulated at multiple levels. For example, the dynamic shuttling of the inactive NFκB dimers between the cytoplasm and nucleus by IκB proteins and its termination by phosphorylation and proteasomal degradation, direct phosphorylation, acetylation of NFκB factors, and dynamic reorganization of NFκB subunits among the activated NFκB dimers have all been identified as key regulatory steps in NFκB activation and, consequently, in NFκB-mediated transcription processes. Thus, an NFκB-regulating peptide is capable of modulating the transcription of genes that are under the control of the NFκB/Rel family of transcription factors. Modulation comprises the upregulation or the downregulation of transcription. In a preferred embodiment, a peptide according to the invention, or a functional derivative or analogue thereof is used for the production of a pharmaceutical composition for the treatment of a parasitic infection or the treatment of an inflammatory condition found after a parasitic infection, such as seen with malaria.

Use of a gene-regulatory peptide or analogue thereof is herein also provided to control many of the inflammatory, allergic and other immune-mediated problems that are caused by parasitic diseases, such as malaria, onchocerciasis, lymphatic filariasis, and trachoma, among others. These inflammatory problems may occur in patients even when they have been successfully treated for the underlying parasitic infection, because the inflammatory response can persist for long periods of time after the parasites have been eliminated. Of course, the need for an anti-inflammatory peptide product is even greater when patients have been infected with parasites that are partially or fully resistant to available anti-parasitic drugs.

The World Health Organization (“WHO”) states that there are an estimated 500 million people in the world who have malaria, with 2 million deaths, each year from the disease—mostly in younger children. Despite strenuous efforts on the part of WHO and other institutions, the disease has been very difficult to control, because mosquitoes become resistant to the insecticides, parasites become resistant to the anti-malarial agents and the inflammatory and immune problems persist even after the parasites are cleared.

Although a gene-regulatory peptide is not expected to kill the parasites that devastate hundreds of millions of people in the developing world, they mediate both (a) the acute, life threatening cytokine-induced inflammatory conditions, and (b) the sub-acute highly debilitating auto-immune and allergic-type reactions, that are associated with many of these parasitic diseases. The parasitic diseases (notably malaria, and especially in malaria caused by P. falciparum) tend to induce cytokines and other immune mediators (such as nitric oxide), triggering SIRS, and resulting in multi-organ dysfunction syndrome (“MODS”), disseminated intravascular coagulation (“DIC”), and multi-organ failure (“MOF”). In other words, many parasites trigger the same reactions from the immune system (i.e., trigger the release of the same mediators) that are triggered by bacterial infections, and can lead to SIRS. Using gene-regulatory peptide therapy as provided herein will significantly slow down the various types of cytokine-driven disease progression—even in those cases where the parasite is susceptible to anti-infective agents, including and especially cerebral malaria.

Sub-acute auto-immune and allergic-type reactions are also induced by parasites. For example, malaria demonstrates the dangerous effects of the auto-immune reactions that can occur (and persist) even when anti-malarial agents have succeeded in clearing the tissues—in this case, red blood cells (“RBCs”)—of parasites. It is not uncommon for millions of RBCs that were never infected by parasites to be destroyed. This hemolysis is due to auto-antibodies and other immune factors, which induce apoptosis (cell death). Thus, even when the parasites have been eradicated by antimalarial drugs, patients often develop anemia that is life-threatening. The threat to life is due not only to the profound anemia, but also to the damage caused to the kidneys by the significant quantities of intracellular contents that develop upon the lysis of vast numbers of RBCs. This problem is particularly acute with respect to the “Blackwater fever” variant of malaria, where the mortality is 20-30%, due to massive intravascular hemolysis leading to acute renal failure, even though parasitemia is absent at the time that hemolysis occurs. Use of an inflammatory peptide product according to the invention affords significant reduction in this rate of mortality.

In many of the parasitic diseases, there are repeat infections over the course of years. Eventually individuals may build up sufficient immunity that the subsequent infections decrease in severity. However, over the course of these repeat infections the intense allergic reactions (to various components of the infecting organism) cause a great deal of tissue destruction. These allergic reactions, which are IgE-mediated, can cause the deposition of IgE-containing immune complexes in (i) small blood vessels, leading to overproduction of TNF-α with all its complications, (e.g. plugging of the blood vessels), and (ii) in the conjuctiva of the eyes, leading to scarring and blindness.

There are several types of interactions between IgE and transcription factor NF-κB (also called NF-κB). The interactions between NF-κB and IgE include the following:

IgE isotype switching is dependent on the binding of NF-κB to a gene promoter.

Aggregation of the high-affinity IgE receptor-on monocytes and dendritic cells leads to synthesis and release of TNF-α and monocyte chemoattractant protein-1.

The parasite products, like endotoxin, induce activation of the cytokine cascade. Cytokine production may also be stimulated by IgE-antigen or IgE-antiIgE complexes. Thus, parasite product and antibody complexes stimulate cells of the macrophage-monocyte series, and possibly endothelium, to release inflammatory cytokines in malaria.

Using a gene-regulatory peptide that is capable of down-regulating inflammatory pathways as provided herein will reduce the extent of the IgE-induced allergic damage and other systemic inflammatory conditions that takes place in many of the chronic/recurring parasitic diseases. Examples of useful NFκB down-regulating peptides to be included in such a pharmaceutical composition are VLPALPQVVC, LQGVLPALPQ, LQG, LQGV, GVLPALPQ, VLPALP, VVC, MTR and circular LQGVLPALPQVVC. More gene-regulating peptides and functional analogues can be found in a (bio)assay, such as an NFκB translocation assay as provided herein. Most prominent among NFκB down-regulating peptides are VLPALPQVVC, LQGVLPALPQ, LQG, LQGV, and VLPALP. These are also capable of reducing production of NO by a cell. Furthermore, LQG, VVC and MTRV, and in particular LQGV promote angiogenesis, especially in topical applications.

It is herein also provided to use a composition that comprises at least two oligopeptides or functional analogues thereof, each capable of down-regulation NFκB, and thereby reducing production of NO and/or TNF-α by a cell, in particular wherein the at least two oligopeptides are selected from the group LQGV, AQGV and VLPALP. Useful NFκB up-regulating peptides are VLPALPQ, GVLPALP and MTRV. As indicated, more gene-regulatory peptides may be found with an appropriate (bio)assay. A gene-regulatory peptide as used herein is preferably short. Preferably, such a peptide is 3 to 15 amino acids long, more preferably, wherein the lead peptide is 3 to 9 amino acids long, most preferred wherein the lead peptide is 4 to 6 amino acids long, and capable of modulating the expression of a gene, such as a cytokine, in a cell. In a preferred embodiment, a peptide is a gene-regulatory peptide that is capable of traversing the plasma membrane of a cell or, in other words, a peptide that is membrane-permeable.

Functional derivative or analogue herein relates to the signaling molecular effect or activity as for example can be measured by measuring nuclear translocation of a relevant transcription factor, such as NF-κB in an NF-κB assay, or AP-1 in an AP-1 assay, or by another method as provided herein. Fragments can be somewhat (i.e. 1 or 2 amino acids) smaller or larger on one or both sides, while still providing functional activity. Such a bioassay comprises an assay for obtaining information about the capacity or tendency of a peptide, or a modification thereof, to regulate expression of a gene. A scan with for example a 15-mer, or a 12-mer, or a 9-mer, or a 8-mer, or a 7-mer, or a 6-mer, or a 5-mer, or a 4-mer or a 3-mer peptides can yield valuable information on the linear stretch of amino acids that form an interaction site and allows identification of gene-regulatory peptides that have the capacity or tendency to regulate gene expression. Gene-regulatory peptides can be modified to modulate their capacity or tendency to regulate gene expression, which can be easily assayed in an in vitro bioassay such as a reporter assay. For example, a particular amino acid at an individual position can be replaced with another amino acid of similar or different properties. Alanine (Ala)-replacement scanning, involving a systematic replacement of each amino acid by an Ala residue, is a suitable approach to modify the amino acid composition of a gene-regulatory peptide when in a search for a gene-regulatory peptide capable of modulating gene expression. Of course, such replacement scanning or mapping can be undertaken with amino acids other than Ala as well, for example with D-amino acids. In one embodiment, a peptide derived from a naturally occurring polypeptide is identified as being capable of modulating gene expression of a gene in a cell. Subsequently, various synthetic Ala-mutants of this gene-regulatory peptide are produced. These Ala-mutants are screened for their enhanced or improved capacity to regulate expression of a gene compared to gene-regulatory polypeptide.

Furthermore, a gene-regulatory peptide, or a modification or analogue thereof, can be chemically synthesized using D- and/or L-stereoisomers. For example, a gene-regulatory peptide that is a retro-inverso of an oligopeptide of natural origin is produced. The concept of polypeptide retro-inversion (assembly of a natural L-amino acid-containing parent sequence in reverse order using D-amino acids) has been applied successfully to synthetic peptides. Retro-inverso modification of peptide bonds has evolved into a widely used peptidomimetic approach for the design of novel bioactive molecules which has been applied to many families of biologically active peptides. The sequence, amino acid composition and length of a peptide will influence whether correct assembly and purification are feasible. These factors also determine the solubility of the final product. The purity of a crude peptide typically decreases as the length increases. The yield of peptide for sequences less than 15 residues is usually satisfactory, and such peptides can typically be made without difficulty. The overall amino acid composition of a peptide is an important design variable. A peptide's solubility is strongly influenced by composition. Peptides with a high content of hydrophobic residues, such as Leu, Val, Ile, Met, Phe and Trp, will either have limited solubility in aqueous solution or be completely insoluble. Under these conditions, it can be difficult to use the peptide in experiments, and it may be difficult to purify the peptide if necessary. To achieve a good solubility, it is advisable to keep the hydrophobic amino acid content below 50% and to make sure that there is at least one charged residue for every five amino acids. At physiological pH Asp, Glu, Lys, and Arg all have charged side chains. A single conservative replacement, such as replacing Ala with Gly, or adding a set of polar residues to the N- or C-terminus, may also improve solubility. Peptides containing multiple Cys, Met, or Trp residues can also be difficult to obtain in high purity partly because these residues are susceptible to oxidation and/or side reactions. If possible, one should choose sequences to minimize these residues. Alternatively, conservative replacements can be made for some residues. For instance, norleucine can be used as a replacement for Met, and Ser is sometimes used as a less reactive replacement for Cys. If a number of sequential or overlapping peptides from a protein sequence are to be made, making a change in the starting point of each peptide may create a better balance between hydrophilic and hydrophobic residues. A change in the number of Cys, Met, and Trp residues contained in individual peptides may produce a similar effect. In another embodiment of the invention, a gene-regulatory peptide capable of modulating gene expression is a chemically modified peptide. A peptide modification includes phosphorylation (e.g., on a Tyr, Ser or Thr residue), N-terminal acetylation, C-terminal amidation, C-terminal hydrazide, C-terminal methyl ester, fatty acid attachment, sulfonation (tyrosine), N-terminal dansylation, N-terminal succinylation, tripalmitoyl-S-Glyceryl Cysteine (PAM3 Cys-OH) as well as farnesylation of a Cys residue. Systematic chemical modification of a gene-regulatory peptide can for example be performed in the process of gene-regulatory peptide optimization.

Synthetic peptides can be obtained using various procedures known in the art. These include solid phase peptide synthesis (SPPS) and solution phase organic synthesis (SPOS) technologies. SPPS is a quick and easy approach to synthesize peptides and small proteins. The C-terminal amino acid is typically attached to a cross-linked polystyrene resin via an acid labile bond with a linker molecule. This resin is insoluble in the solvents used for synthesis, making it relatively simple and fast to wash away excess reagents and by-products.

The peptides as mentioned in this document such as LQG, AQG, LQGV, AQGV, LQGA, VLPALP, ALPALP, VAPALP, ALPALPQ, VLPAAPQ, VLPALAQ, LAGV, VLAALP, VLPALA, VLPALPQ, VLAALPQ, VLPALPA, GVLPALP, VVCNYRDVRFESIRLPGCPRGVNPYVSYAVALSCQCAL, RPRCRPINATLAVEKEGCPVCITVNTTICAGYCPT, SKAPPPSLPSPSRLPGPS, LQGVLPALPQVVC, SIRLPGCPRGVNPVVS, LPGCPRGVNPVVS, LPGC, MTRV, MTR, and VVC were prepared by solid-phase synthesis using the fluorenylmethoxycarbonyl (Fmoc)/tert-butyl-based methodology with 2-chlorotrityl chloride resin as the solid support. The side-chain of glutamine was protected with a trityl function. The peptides were synthesized manually. Each coupling consisted of the following steps: (i) removal of the α-amino Fmoc-protection by piperidine in dimethylformamide (DMF), (ii) coupling of the Fmoc amino acid (3 eq) with diisopropylcarbodiimide (DIC)/1-hydroxybenzotriazole (HOBt) in DMF/N-methylformamide (NMP) and (iii) capping of the remaining amino functions with acetic anhydride/diisopropylethylamine (DIEA) in DMF/NMP. Upon completion of the synthesis, the peptide resin was treated with a mixture of trifluoroacetic acid (TFA)/H₂O/triisopropylsilane (TIS) 95:2.5:2.5. After 30 minutes TIS was added until decolorization. The solution was evaporated in vacuo and the peptide precipitated with diethyl ether. The crude peptides were dissolved in water (50-100 mg/ml) and purified by reverse-phase high-performance liquid chromatography (RP-HPLC). HPLC conditions were: column: Vydac TP21810C18 (10×250 mm); elution system: gradient system of 0.1% TFA in water v/v (A) and 0.1% TFA in. acetonitrile (ACN) v/v (B); flow rate 6 ml/minute; absorbance was detected from 190-370 nm. There were different gradient systems used. For example for peptides LQG and LQGV: 10 minutes 100% A followed by linear gradient 0-10% B in 50 minutes. For example for peptides VLPALP and VLPALPQ: 5 minutes 5% B followed by linear gradient 1% B/minute. The collected fractions were concentrated to about 5 ml by rotation film evaporation under reduced pressure at 40° C. The remaining TFA was exchanged against acetate by eluting two times over a column with anion exchange resin (Merck II) in acetate form. The elute was concentrated and lyophilized in 28 hours. Peptides later were prepared for use by dissolving them in PBS.

RAW 264.7 macrophages, obtained from American Type Culture Collection (Manassas, Va.), were cultured at 37° C. in 5% CO2 using DMEM containing 10% FBS and antibiotics (100 U/ml of penicillin, and 100 μg/ml streptomycin). Cells (1×10⁶/ml) were incubated with peptide (10 μg/ml) in a volume of 2 ml. After 8 hours of cultures, cells were washed and prepared for nuclear extracts.

Nuclear extracts and EMSA were prepared according to Schreiber et al. Methods (Schreiber et al. 1989, Nucleic Acids Research 17). Briefly, nuclear extracts from peptide stimulated or nonstimulated macrophages were prepared by cell lysis followed by nuclear lysis. Cells were then suspended in 400 μl of buffer (10 mM HEPES (pH 7.9), 10 mM KCl, 0.1 mM KCL, 0.1 mM EDTA, 0.1 mM EGTA, 1 mM DTT, 0.5 mM PMSF and protease inhibitors), vigorously vortexed for 15 seconds, left standing at 4° C. for 15 minutes, and centrifuged at 15,000 rpm for 2 minutes. The pelleted nuclei were resuspended in buffer (20 mM HEPES (pH 7.9), 10% glycerol, 400 mM NaCl, 1 mM EDTA, 1 mM EGTA, 1 mM DTT, 0.5 mM PMSF and protease inhibitors) for 30 minutes on ice, then the lysates were centrifuged at 15,000 rpm for 2 minutes. The supernatants containing the solubilized nuclear proteins were stored at −70° C until used for the Electrophoretic Mobility Shift Assays (EMSA).

Electrophoretic mobility shift assays were performed by incubating nuclear extracts prepared from control (RAW 264.7) and peptide treated RAW 264.7 cells with a 32P-labeled double-stranded probe (5′ AGCTCAGAGGGGGACTTTCCGAGAG 3′) synthesized to represent the NF-κB binding sequence. Shortly, the probe was end-labeled with T4 polynucleotide kinase according to manufacturer's instructions (Promega, Madison, Wis.). The annealed probe was incubated with nuclear extract as follows: in EMSA, binding reaction mixtures (20 μl) contained 0.25 μg of poly(dI-dC) (Amersham Pharmacia Biotech) and 20,000 rpm of 32P-labeled DNA probe in binding buffer consisting of 5 mM EDTA, 20% Ficoll, 5 mM DTT, 300 mM KCl and 50 mM HEPES. The binding reaction was started by the addition of cell extracts (10 μg) and was continued for 30 minutes at room temperature. The DNA-protein complex was resolved from free oligonucleotide by electrophoresis in a 6% polyacrylamide gel. The gels were dried and exposed to x-ray films.

The transcription factor NF-κB participates in the transcriptional regulation of a variety of genes. Nuclear protein extracts were prepared from LPS and peptide treated RAW264.7 cells or from LPS treated RAW264.7 cells. In order to determine whether the peptide modulates the translocation of NF-κB into the nucleus, on these extracts EMSA was performed. Here we see that indeed some peptides are able to modulate the translocation of NF-κB since the amount of labeled oligonucleotide for NF-κB is reduced. In this experiment peptides that show the modulation of translocation of NF-κB are: VLPALPQVVC, LQGVLPALPQ, LQG, LQGV, GVLPALPQ, VLPALP, VLPALPQ, GVLPALP, VVC, MTRV, MTR.

RAW 264.7 mouse macrophages were cultured in DMEM, containing 10% or 2% FCS, penicillin, streptomycin and glutamine, at 37° C., 5% CO₂. Cells were seeded in a 12-wells plate (3×10⁶ cells/ml) in a total volume of 1 ml for 2 hours and then stimulated with LPS (E. coli 026:B6; Difco Laboratories, Detroit, Mich.) and/or gene-regulatory peptide (1 microgram/ml). After 30 minutes of incubation, plates were centrifuged and cells were collected for nuclear extracts. Nuclear extracts and EMSA were prepared according to Schreiber et al. Cells were collected in a tube and centrifuged for 5 minutes at 2000 rpm (rounds per minute) at 4° C. (Universal 30 RF, Hettich Zentrifuges). The pellet was washed with ice-cold Tris buffered saline (TBS pH 7.4) and resuspended in 400 μl of a hypotonic buffer A (10 mM HEPES pH 7.9, 10 mM KCl, 0.1 mM EDTA, 0.1 mM EGTA, 1 mM DTT, 0.5 mM PMSF and protease inhibitor cocktail (Complete™ Mini, Roche) and left on ice for 15 minutes. Twenty-five micro liter 10% NP-40 was added and the sample was centrifuged (2 minutes, 4000 rpm, 4° C.). The supernatant (cytoplasmic fraction) was collected and stored at −70° C. The pellet, which contains the nuclei, was washed with 50 μl buffer A and resuspended in 50 μl buffer C (20 mM HEPES pH 7.9, 400 mM NaCl, 1 mM EDTA, 1 mM EGTA, 1 mM DTT, 0.5 mM PMSF and protease inhibitor cocktail and 10% glycerol). The samples were left to shake at 4° C. for at least 60 minutes. Finally the samples were centrifuged and the supernatant (nucleic fraction) was stored at −70° C.

Bradford reagent (Sigma) was used to determine the final protein concentration in the extracts. For electrophoretic mobility shift assays (EMSA) an oligonucleotide representing NF-κB binding sequence (5′-AGC TCA GAG GGG GAC TTT CCG AGA G-3′) was synthesized. Hundred pico mol sense and antisense oligo were annealed and labeled with γ-³²P-dATP using T4 polynucleotide kinase according to manufacturer's instructions (Promega, Madison, Wis.). Nuclear extract (5-7.5 μg) was incubated for 30 minutes with 75000 cpm probe in binding reaction mixture (20 microliter) containing 0.5 μg poly dI-dC (Amersham Pharmacia Biotech) and binding buffer BSB (25 mM MgCl₂, 5 mM CaCl₂, 5 mM DTT and 20% Ficoll) at room temperature. The DNA-protein complex was resolved from free oligonucleotide by electrophoresis in a 4-6% polyacrylamide gel (150 V, 2-4 hours). The gel was then dried and exposed to x-ray film. The transcription factor NF-κB participates in the transcriptional regulation of a variety of genes. Nuclear protein extracts were prepared from either LPS (1 mg/ml), peptide (1 mg/ml) or LPS in combination with peptide treated and untreated RAW264.7 cells. In order to determine whether the peptides modulate the translocation of NF-κB into the nucleus, on these extracts EMSA was performed. Peptides are able to modulate the basal as well as LPS induced levels of NF-κB. In this experiment peptides that show the inhibition of LPS induced translocation of NF-κB are: VLPALPQVVC, LQGVLPALPQ, LQG, LQGV, GVLPALPQ, VLPALP, VVC, MTR and circular LQGVLPALPQVVC. Peptides that in this experiment promote LPS induced translocation of NF-κB are: VLPALPQ, GVLPALP and MTRV. Basal levels of NF-κB in the nucleus was decreased by VLPALPQVVC, LQGVLPALPQ, LQG and LQGV while basal levels of NF-κB in the nucleus was increased by GVLPALPQ, VLPALPQ, GVLPALP, VVC, MTRV, MTR and LQGVLPALPQVVC. In other experiments, QVVC also modulated the translocation of NF-κB into the nucleus (data not shown).

Further modes of identification of gene-regulatory peptides by NFκB analysis

Cells: Cells will be cultured in appropriate culture medium at 37° C., 5% CO₂. Cells will be seeded in a 12-wells plate (usually 1×10⁶ cells/ml) in a total volume of 1 ml for 2 hours and then stimulated with regulatory peptide in the presence or absence of additional stimuli such as LPS. After 30 minutes of incubation plates will be centrifuged and cells are collected for cytosolic or nuclear extracts.

Nuclear Extracts: Nuclear extracts and EMSA could be prepared according to Schreiber et al. Method (Schreiber et al. 1989, Nucleic Acids Research 17). Cells are collected in a tube and centrifuged for 5 minutes at 2000 rpm (rounds per minute) at 4° C. (Universal 30 RF, Hettich Zentrifuges). The pellet is washed with ice-cold Tris buffered saline (TBS pH 7.4) and resuspended in 400 μl of a hypotonic buffer A (10 mM HEPES pH 7.9, 10 mM KCl, 0.1 mM EDTA, 0.1 mM EGTA, 1 mM DTT, 0.5 mM PMSF and protease inhibitor cocktail (Complete™ Mini, Roche) and left on ice for 15 minutes. Twenty-five micro liter 10% NP-40 is added and the sample is centrifuged (2 minutes, 4000 rpm, 4° C.). The supernatant (cytoplasmic fraction) was collected and stored at −70° C. for analysis. The pellet, which contains the nuclei, is washed with 50 μl buffer A and resuspended in 50 μl buffer C (20 mM HEPES pH 7.9, 400 mM NaCl, 1 mM EDTA, 1 mM EGTA, 1 mM DTT, 0.5 mM PMSF and protease inhibitor cocktail and 10% glycerol). The samples are left to shake at 4° C. for at least 60 minutes. Finally the samples are centrifuged and the supernatant (nucleic fraction) is stored at −70° C. for analysis.

Bradford reagent (Sigma) could be used to determine the final protein concentration in the extracts.

EMSA: For Electrophoretic mobility shift assays an oligonucleotide representing NF-κB binding sequence such as (5′-AGC TCA GAG GGG GAC TTT CCG AGA G-3′) are synthesized. Hundred pico mol sense and antisense oligo are annealed and labeled with γ-³²P-dATP using T4 polynucleotide kinase according to manufacture's instructions (Promega, Madison, Wis.). Cytosolic extract or nuclear extract (5-7.5 μg) from cells treated with regulatory peptide or from untreated cells is incubated for 30 minutes with 75000 cpm probe in binding reaction mixture (20 μl) containing 0.5 μg poly dI-dC (Amersham Pharmacia Biotech) and binding buffer BSB (25 mM MgCl₂, 5 mM CaCl₂, 5 mM DTT and 20% Ficoll) at room temperature. Or cytosolic and nuclear extract from untreated cells or from cells treated with stimuli could also be incubated with probe in binding reaction mixture and binding buffer. The DNA-protein complex is resolved from free oligonucleotide by electrophoresis in a 4-6% polyacrylamide gel (150 V, 2-4 hours). The gel is then dried and exposed to x-ray film. Peptides can be biotinylated and incubated with cells. Cells are then washed with phosphate-buffered saline, harvested in the absence or presence of certain stimulus (LPS, PHA, TPA, anti-CD3, VEGF, TSST-1, VIP or know drugs etc.). After culturing cells are lysed and cells lysates (whole lysate, cytosolic fraction or nuclear fraction) containing 200 micro gram of protein are incubated with 50 miroliter Neutr-Avidin-plus beads for 1 hour at 4° C. with constant shaking. Beads are washed five times with lysis buffer by centrifugation at 6000 rpm for 1 minute. Proteins are eluted by incubating the beads in 0.05 N NaOH for 1 minute at room temperature to hydrolyze the protein-peptide linkage and analyzed by SDS-polyacrylamide gel electrophoresis followed by immunoprecipitated with agarose-conjugated anti-NF-κB subunits antibody or immunoprecipitated with antibody against to be studied target. After hydrolyzing the protein-peptide linkage, the sample could be analyzed on HPLS and mass-spectrometry. Purified NF-κB subunits or cell lysate interaction with biotinylated regulatory peptide can be analyzed on biosensor technology. Peptides can be labeled with FITC and incubated with cells in the absence or presence of different stimulus. After culturing, cells can be analyzed with fluorescent microscopy, confocal microscopy, flow cytometry (cell membrane staining and/or intracellular staining) or cells lysates are made and analyzed on HPLC and mass-spectrometry. NF-κB transfected (reporter gene assay) cells and gene array technology can be used to determine the regulatory effects of peptides.

HPLC and mass-spectrometry analysis: Purified NF-κB subunit or cytosolic/nuclear extract is incubated in the absence or presence of (regulatory) peptide is diluted (2:1) with 8 N guanidinium chloride and 0.1% trifluoroacetic acid, injected into a reverse-phase HPLC column (Vydac C18) equilibrated with solvent A (0.1% trifluoroacetic acid), and eluted with a gradient of 0 to 100% eluant B (90% acetonitrile in solvent A). Factions containing NF-κB subunit are pooled and concentrated. Fractions are then dissolved in appropriate volume and could be analyzed on mass-spectrometry.

Further references:

PCT International Publications WO99/59617, WO01/72831, WO97/49721, WO01/10907, and WO01/11048, the contents of the entirety of all of which are hereby incorporated by this reference.

What is claimed is:

1. A pharmaceutical composition for topical application, said pharmaceutical composition comprising:
a gene-regulatory peptide or functional analogue thereof.

2. The pharmaceutical composition of claim 1 wherein said gene-regulatory peptide or functional analogue thereof modulates translocation, activity, or translocation and activity of a gene transcription factor.

3. The pharmaceutical composition of claim 2 wherein said gene transcription factor comprises an NF-kappaB/Rel protein.

4. The pharmaceutical composition of claim 3 wherein translocation, activity, or translocation and activity of said NF-kappaB/Rel protein is inhibited.

5. A composition for topical administration of a peptide to a subject, said pharmaceutical composition comprising:
a pharmacologically effective amount of a gene-regulatory peptide thereof that modulates translocation, activity, or translocation and activity of a gene transcription factor, and
a pharmaceutically acceptable vehicle or system for delivering said gene-regulatory peptide or functional analogue thereof.

6. The composition of claim 5 wherein said gene transcription factor is an NF-kappaB/Rel protein.

7. The composition of claim 6 wherein translocation, activity, or translocation and activity of said NF-kappaB/Rel protein is inhibited.

8. The composition of claim 5 wherein said composition has a form of a gel, solution, lotion, balm, transdermal patch, or ointment.
